(19) United States
(12) Patent Application Publication
(10) Pub. No.: US 2021/0358617 A1
(43) Pub. Date: Nov. 18, 2021

(54) ON-BODY SENSOR SYSTEM

(71) Applicant: KONINKLIJKE PHILIPS N.V., EINDHOVEN (NL)

(72) Inventors: Ashoka Sathanur VISWESWARA, Amstelveen (NL); Mark Thomas JOHNSON, Arendonk (BE); Mohammed MEFTAH, Tilburg (NL)

(21) Appl. No.: 17/286,139

(22) PCT Filed: Oct. 15, 2019
(86) PCT No.: PCT/EP2019/077903

§ 371 Date: Apr. 16, 2021

Related U.S. Application Data
(60) Provisional application No. 62/746,172, filed on Oct. 16, 2018.

(51) Int. Cl. G16H 40/67; G16H 40/40; A61B 5/00

(52) U.S. Cl. CPC G16H 40/67; A61B 5/0028; A61B 5/684; G16H 40/40

(57) ABSTRACT

An on-body sensor system (30) comprises at least two skin interface units (32, 34) for coupling signals into and out of the body, with one of the units being for placement at a known location. One unit applies electrical signals to the body and the other senses them at a remote location. By analyzing the sensed signals using a set of pre-determined body-transmission parameters, a position of one of the skin interface units can be determined. This allows accurate placement of one or the units, for instance to allow more accurate monitoring of physiological parameters using the unit. The body transmission parameters can change over time, whereas once the interface units are put in position, their position is stable. Hence the system also includes functionality to re-calibrate the transmission parameters using at least one known stable set of initial positions of the interface units. The re-calibration comprises a process of re-calculating the parameters based on the known positions. These can then be stored and used for future determinations of the position of the one of the skin interface units having a moveable location, for instance in the case that it is re-positioned or replaced.

### FIELD OF THE INVENTION

The invention relates to an on-body sensor system having means for establishing a position of one or more on-body sensor elements.

### BACKGROUND OF THE INVENTION

On-body sensing systems permit accurate long-term monitoring of physiological parameters of a subject. On-body systems are based on use of wearable devices or units, including for instance patches, which are mountable on the body and maintain a stable position over time. By electrically interfacing with the skin or body, vital signs or other parameters can be monitored.

On-body systems may typically be used in low acuity settings such as a general ward and also at a subject's home. Improved reliability in physiological parameter monitoring in general wards is needed to reduce mortality rates, by enabling detection of any deterioration in condition as early as possible. The capacity to monitor reliably at a subject's home also permits earlier discharge of patients without risk of undetected deterioration. Monitoring will typically continue up to 30 days from discharge for example.

In the case of patches, in many cases these need to be changed every 2-3 days because of depleted battery charge, degradation of adhesion or skin irritation. As a result, it falls to a patient themselves or an informal care giver such as a relative to replace and re-attach the patch. In some cases, the patch has to be moved to other alternative locations and orientations. Accurate placement of the patch upon replacement is important to ensure that physiological parameters are correctly determined.

Methods have been proposed to permit determination of a position of an on-body element such as a patch. This can be used to guide a user in correctly positioning the element on the body.

One approach is based on application of an electrical field model of the human body. The model can be used to determine the frequency response of the human body as a signal transmission medium. It is measured by generating and capacitively coupling an electrical signal having a known frequency and amplitude at one point on the human body. The coupled signal is then sensed and measured at a different, remote point on the body by a sensor. The received signal is analyzed and various signal properties derived. This process can be repeated for multiple different signals having different transmitter frequencies and also for various distances and body locations of the on-body sensing element relative to the transmitting location.

One example model is presented in Namjum Cho, et al. (2007). The Human Body Characteristics as a Signal Transmission Medium for Intrabody Communication. IEEE Transactions on Microwave Theory and Techniques. In this paper, the authors propose a near-field coupling model of the human body based on modelling the human body in terms of three cylinders: two for the arms and one for the human torso. This is illustrated in FIG. 1.

As shown in FIG. 1(a), the arms and the human torso are segmented with 10 cm long unit blocks, each with resistances and capacitances. The arms and the torso are together modelled as distributed RC network as shown in FIG. 1(b). In a similar manner, a human leg may be modelled with corresponding resistances and impedances. The arm model has resistance and capacitance with subscript “A” and the torso has resistance and capacitance with subscript “T”.

One practical implementation of the signal transmission and sensing approach is presented in Zhang, Y. et al, 2016, May. “Skintrack: Using the body as an electrical waveguide for continuous finger tracking on the skin”. In Proceedings of the 2016 CHI Conference on Human Factors in Computing Systems (pp. 1491-1503).

This paper proposes a continuous finger tracking technology called SkinTrack. SkinTrack is a wearable system that enables continuous touch tracking across the skin. The system comprises a ring, which emits a continuous high-frequency AC signal, and a sensing wristband embodying multiple sensor electrodes. Due to the phase delay inherent in propagating the high frequency AC signal through the body, a phase difference can be observed between pairs of electrodes. The SkinTrack system measures these phase differences to compute a 2D coordinate location of the subject's finger touching on their skin. The resolution (i.e. accuracy) of SkinTrack method is approximately 7 mm.

The same paper describes a method whereby the phase angle difference between the sensed signals at two different locations on the human body is used as a measure of localization of the signal transmitter with respect to the sensors. FIG. 2 schematically illustrates the technique, where the transmitter is in the form of a ring 12. The location of the transmitter relative to two sensor electrodes 14a, 14b on a smartwatch is identified using the technique.

When an 80 MHz RF signal is used, the wavelength of the electromagnetic wave propagating through the human body is around 91 cm. This results in phase angle difference of approximately 4°/cm for one single cycle of the wave. If the localization is performed within one wavelength of the RF signal (i.e. within around 91 cm), then it is possible to uniquely locate the position of the transmitter with respect to the two sensors by measuring the phase angle difference between the two received signals.

The RF signal propagation characteristics across skin vary over time. This variation is due to environmental factors which result in changes in moisture level of the skin. This change in skin-transmission characteristics (otherwise known as skin channel characteristics) results in a change in various parameters associated with the signal propagation, including the signal propagation velocity and, as a consequence, the signal wavelength. This change in signal wavelength results in change of various signal parameters of interest such as phase angle difference value, time of flight value (signal transmission time) and signal path loss value (signal attenuation). These parameters are necessary however for accurate determination of position and orientation of the transmitter on the body.

### SUMMARY OF THE INVENTION

The invention is defined by the claims.

It has been realized by the inventors that in practical applications of on-body sensing systems, this variation in skin characteristics will cause complications in cases in which the on-body element (such as a patch) needs to be changed by the patient at home, as discussed above. Accurate sensing of the real-time position of the element is important to enable the system to guide the user in placing the element in the correct position. However, if the skin transmission characteristics have changed between the time the system was initially calibrated at the hospital, and the time the patient changes the patch, the measured signal properties such as phase angle difference, time of flight and path loss values also change. This will lead to inaccurate determination of position of the transmitter and so inaccurate guidance as to the correct positioning of the transmitter. This will lead to an incorrect placement of the on-body element, which will affect the reliability of the physiological parameter monitoring.

The present invention aims to address the above problems.

According to examples in accordance with an aspect of the invention, there is provided an on-body sensor system, comprising:
at least two skin interface units for electrically interfacing with the skin of a subject, including a first unit for coupling generated signals into the body, and a second unit for sensing said coupled signals at a remote location on the subject's skin, one of the units for placement at a known location on the body; and

a controller adapted to control signal generation and sensing using the skin interface units, and operable in one control mode to determine an indication of a position of one of the units based on sensed signal characteristics at said second unit and one or more pre-determined body-transmission parameters;

wherein the controller is operable in a further control mode to perform a re-calibration procedure for re-determining said body-transmission parameters based on a known initial position of the two units, the procedure comprising
controlling the first skin interface unit to generate one or more reference signals,
sensing the reference signals at the second skin interface unit and re-determining at least one of the body-transmission parameters based on the sensed signal characteristics and said known initial position of the two units, and
correcting the pre-determined body-transmission parameters based on any differences between the at least one re-determined parameter and the corresponding pre-determined parameter.
The invention proposes a sensor system which has a pair of skin interface units having means for electrically coupling with the skin to transmit and receive signals through the body. Each may comprise one or more electrode pairs for coupling signals into and from the body. The system can determine an indication of a position of one of the skin interface units based on characteristics of transmitted signals after they have passed from one unit to the other through the body, and based on a known position of one of the units on the body. Certain body-transmission parameters are also used to do this, for instance relating to a propagation wavelength and velocity through the body, or simply relating to an expected transmission time, phase angle difference and/or attenuation of the signal for different particular positions in which the unit having position to be determined might be placed (permitting position indication to be derived based on a comparison with these).

To overcome the problem of changing body-transmission parameters (between initial placement of a skin interface unit and its subsequent replacement or re-positioning), the system is further adapted to perform a re-calibration procedure. This procedure enables the body-transmission parameters to be re-calculated. The invention is based on the insight that this procedure can always be done in advance of removing and repositioning the interface unit(s). This means that there is always a known starting position of the interface units which can be relied upon (based for instance on the position determined on last placing the units, before the parameters drifted, or based on a known accurate placement by a clinician), and this information can be used to thereby retroactively determine new updated body-transmission parameters.

Hence the invention is based on dynamically determining which information is relied upon as accurate and which is to be re-calculated. Once a unit has been put in place and its position calculated, this position may be stored and assumed known. This can then be used in advance of removing the unit again (i.e. while its position hasn't changed) to re-calculate the transmission parameters. Once the transmission parameters have been re-calculated, these can be stored and assumed known, and can then be used to re-determine an indication of the position of the unit after its replacement. The invention is hence based on dynamically and intelligently adapting its deployment of information to permit two different and interdependent physical variables, each of which may alter, to be determined and kept accurate.

The invention makes use of a controller. The controller may be a separate (dedicated) controller or the control function may be performed by one or both of the skin interface units themselves. Hence, in the latter case, the controller is a distributed controller. Hence one or both of the skin interface units may comprise the controller in some examples, i.e. the control function is distributed among the skin interface units of the system itself. In all explanations and descriptions above and below, reference to a controller may be taken to refer to either a dedicated control unit or to one or more of the interface units of the system performing the relevant control function.

The position of an interface unit may refer to a positioning on the body, or on the skin. Position may mean a relative position between the units, e.g. a distance or separation. Position may also include an orientation (e.g. relative to the skin) as well as a location on the body/skin.

The derived indication of position may be a direct or indirect indication of position. It may be quantitative co-ordinate position for instance, or may comprise simply a set of sensed signal characteristics or parameters which together uniquely characterize the location. This by itself is useful where for example such parameters are compared with previously calculated parameters for different positions (as will be described below). These previously calculated parameters may be the pre-determined body transmission parameters.

The controller may be operable after determining an indication of a position of the one skin interface unit to generate output information representative of, or based on, this determination. This may comprise guidance instructions for guiding a user in placing the unit in a target position.

The re-calibration procedure comprises re-determining at least one of the body-transmission parameters. This re-determining may be based on a pre-stored control routine for example. This may include a pre-determined set of steps to carry out.

The pre-determined body-transmission parameters may be pre-stored, e.g. in a memory, or the parameters may be obtained e.g. from a remote data source such as a remote computer or memory.

The system comprises skin interface units for electrically coupling signals into and back from the skin or body. Each may comprise at least one pair of electrodes for electrically interacting or coupling with the skin, or a different signal coupling means may be used. Each unit is preferably for mounting or applying against the skin, either in contact with the skin or in close proximity to it, possibly separated by a small clearance or space.

One or both of the skin interface units may comprise or consist of a pad, e.g. a patch, for mounting against the skin.

A ‘signal’ means an electrical signal, and may for instance be capacitively coupled into the body, or inductively coupled into the body. The same or a different coupling mechanism may be used to couple signals out of the body for sensing.

The controller may generate signals and use the first skin interface unit to apply these signals to the body. The controller may use the second skin interface unit to sense the same signals at a location remote (i.e. separated) from the first skin interface unit. Alternatively, signal generation and analysis may be performed locally at the interface units themselves. In either case, preferably the signals generated for coupling into the body are in the RF frequency range 10 MHz to 150 MHz since in this frequency range the body acts as a waveguide for signal transmission.

One or both of the skin interface units may comprise a plurality of pairs of skin interfacing electrodes, e.g. skin contacting electrodes.

The system has at least two skin interface units, at least one for generating and transmitting signals, and another for sensing the signals at a remote location. The first and second interface units may be functionally interchangeable, i.e. each can act as either signal generator or signal receiver/sensor. The two may be structurally the same.

Alternatively, the first and second skin interface units may be different in terms of their mounting configuration on the body, and in terms of their broader functionality. In various cases, this can assist in simplifying procedures of determining location or transmission parameters, as will be explained below.

One of the skin interface units is for mounting at a known location on the body, and one of the interface units has a variable position which the controller needs to determine. The known location of the one of the units is used in combination with measured signal characteristics, and the pre-determined transmission parameters to determine the position of the other of the units.

For example, the first interface unit may have a known location. In this case, in the corresponding control mode, the controller is configured to determine a position of the second interface unit. Furthermore, the re-calibration procedure may be based on the known (static) location of the first unit, and a known initial position of the second unit.

To facilitate this, one or both of the skin-interface units may be in the form of a body-mountable unit, for example a sensor patch or wearable device.

In certain examples the first interface unit may be in the form of an on-body unit for (fixedly) mounting against a pre-determined region of the skin of the subject, or may for example be in the form of an off-body unit for temporary placement against a pre-determined region of the skin of the subject.

For example, the first interface unit may a unit shaped to fit to a particular part of the body, e.g. a wearable unit such a watch, ring, arm or ankle band or ear hook for example.

For example, the first interface unit may be a wearable unit configured for mounting to a particular part of the body, for example a wrist mountable unit.

The system uses a set of one or more body-transmission parameters. These may relate to properties of the body or skin as a medium for carrying electrical signals, i.e. the generated signals. They may alternatively relate to properties of the sensed signals (after their propagation through the skin or body), these properties being derivable based on measured characteristics of the signals at the second interface unit.

The one or more body transmission parameters may include for example at least one of: signal wavelength, signal propagation velocity, phase angle difference (between two electrode pairs on a single skin-interface unit), signal transmission time (between signal generation and receipt), and signal attenuation (between signal generation and receipt).

In one set of advantageous examples, the pre-determined transmission parameters may include a plurality of sets of transmission parameters, each set corresponding to a different particular possible position and optionally orientation of the skin interface unit whose position is determined by the controller.

In this case, determining the position of said at least one skin interface unit merely requires analyzing signals sensed at the second interface unit, determining transmission parameters associated with the sensed signal (e.g. deriving these based on measured signal characteristics), and then simply comparing these with each of the sets of pre-determined parameters, to determine which set the measured parameters are most similar to. This then indicates that the at least one interface unit has a current position close to or matching the position associated with said given pre-determined set.

This is a simpler approach than for example calculating a position from first principles using e.g. known signal velocity or wavelength, and measuring signal transmission time.

The controller may in examples be operable in accordance with one control mode to perform an initial calibration procedure to determine and store said predetermined transmission parameters, based on measured signal characteristics at the second skin interface unit with the two units placed in at least one known set of locations. In advantageous examples, the initial calibration procedure may comprise determining and storing a plurality of sets of transmission parameters, each set corresponding to a different particular position and optionally orientation of the skin interface unit whose position the controller is operable to determine. A user may for example move one of the units, for instance the second unit, between different positions and/or orientations while the other unit remains at a fixed known location, with the controller configured to determine and store transmission parameters for each. The controller may be adapted to receive a user input command to indicate when the unit has been moved to each next position.

According to one or more embodiments, the controller may be adapted in accordance with one control mode to guide a user in positioning one of the units based on determining an indication of a current position of the unit using the sensed signal characteristics and the stored body-transmission parameters. In some cases, the determined position indication may be compared a pre-determined target position to derive the guidance.

In accordance with any embodiment of this invention, the system may be for monitoring one or more physiological parameters of a subject, for example vital signs of the subject. In this case, the second interface unit may be for use in sensing the one or more physiological parameters.

Examples in accordance with a further aspect of the invention provide a method of configuring an on-body sensor system,

the system comprising at least two skin interface units for electrically interfacing with the skin of a subject, including a first unit for coupling generated signals into the body, and a second unit for sensing said coupled signals at a remote location on the subject's skin, one of the units for placement at a known location on the body,

and the system operable to determine an indication of position of one of the units based on sensed signal characteristics at said second unit and one or more pre-determined body-transmission parameters,

and the method comprising

executing a re-calibration procedure for re-determining the one or more body-transmission parameters based on a known initial position of the two units, the procedure comprising
controlling the first skin interface unit to generate one or more reference signals, and sensing the reference signals at the second skin interface unit,
re-determining at least one of the body-transmission parameters based on the sensed signal characteristics and said known initial position of the two units, and
correcting the pre-determined body-transmission parameters based on any differences between the at least one re-determined parameter and the corresponding pre-determined parameter.

The recalibration procedure may be performed in advance of re-positioning one of the skin-interface units, for instance the second interface unit. This means that the body-transmission parameters are corrected while an accurate position both of the interface units is known. This known set of positions can be used in the re-calibration procedure.

Accordingly, in accordance with one or more embodiments, the configuration method may further comprise, subsequent to the re-calibration procedure,

re-positioning one of the interface units on the body (the interface unit having a position to be determined by the controller), for instance the second interface unit, and

determining a position of the re-positioned interface unit based on signal characteristics sensed at the second interface unit and the corrected body-transmission parameters.

Re-positioning the interface unit may be performed when the unit needs to be changed for instance. For example, in the case that one or both of the interface units are patches or pads, these may need to be frequently replaced at home by a user. This will typically lead to a slight repositioning when the user then reattaches the new patch or pad.

Furthermore, in accordance with further embodiments, the method may further comprise performing, in advance of the re-calibration procedure, an initial calibration procedure to determine and store said predetermined transmission parameters, based on measured signal characteristics at the second skin interface unit with the two units placed in at least one known set of initial locations, and preferably where this procedure comprises determining and storing a plurality of sets of transmission parameters, each set corresponding to a different particular position and optionally orientation of the skin interface unit having a position to be determined.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIGS. 1a and 1b schematically depicts a near field coupling model of the human body according to the prior art;

FIG. 2 schematically depicts a prior art technique for determining on-body position based on body-transmitted AC signals;

FIG. 3 shows in block diagram form an example system in accordance with one or more embodiments;

FIG. 4 shows in block diagram form an example signal transceiver as may be used in example systems according to one or more embodiments;

FIG. 5 illustrates the arrangement of an example system according to one or more embodiments;

FIG. 6 schematically illustrates an example system according to an embodiment; and

FIG. 7 is a block diagram of an example calibration and re-calibration procedure as may be implemented using a system according to one or more embodiments.

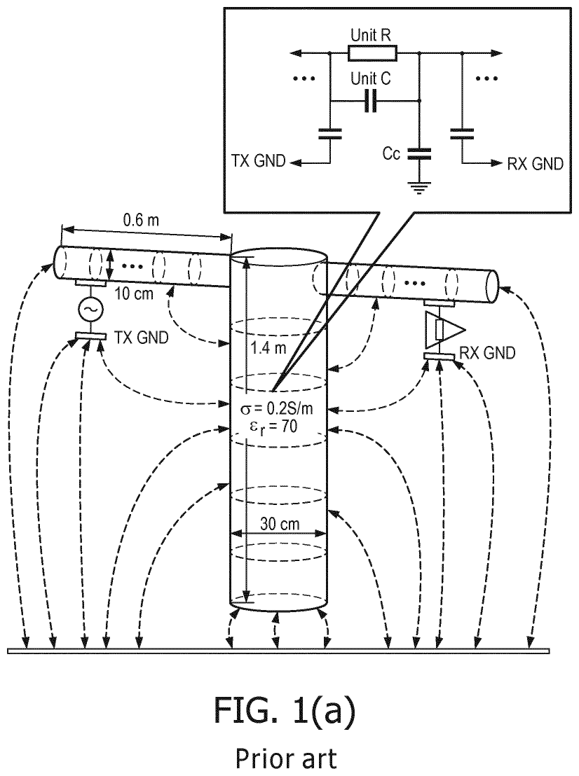

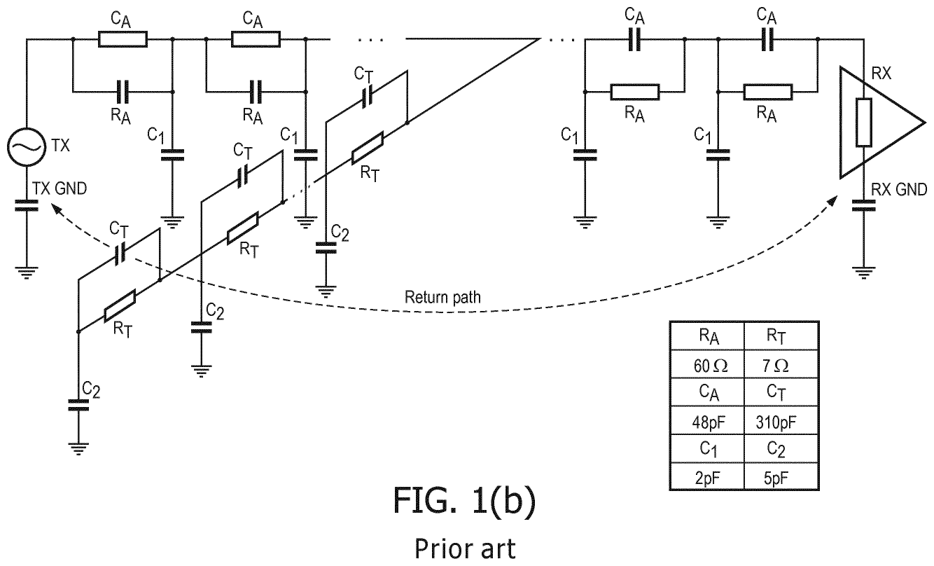

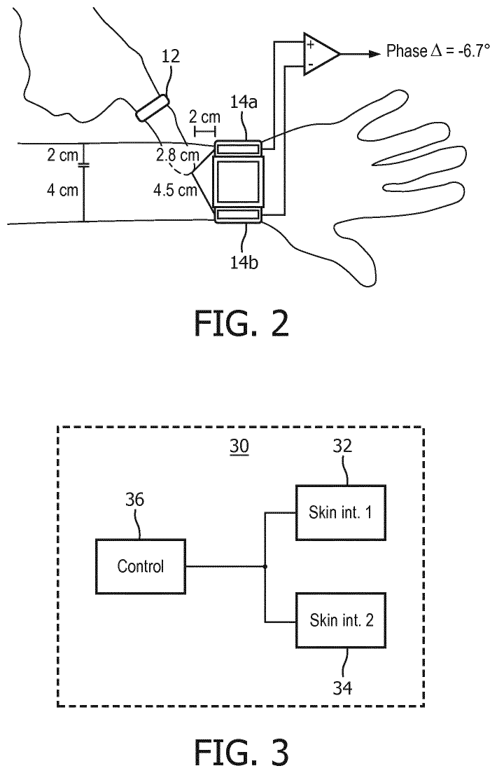

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides an on-body sensor system comprising at least two skin interface units for coupling signals into and out of the body, with one of the units being for placement at a known location. One unit applies electrical signals to the body and the other senses them at a remote location. By analyzing the sensed signals using a set of pre-determined body-transmission parameters, a position of one of the skin interface units can be determined. This allows accurate placement of one of the units, for instance to allow more accurate monitoring of physiological parameters using the unit. The body transmission parameters can change over time, whereas once the interface units are put in position, their position is stable. Hence the system also includes functionality to re-calibrate the transmission parameters using at least one known stable set of initial positions of the interface units. The re-calibration comprises a process of re-calculating the parameters based on the known positions. These can then be stored and used for future determinations of the position of the one of the skin interface units having a moveable location, for instance in the case that it is re-positioned or replaced.

The invention is aimed at allowing an on-body sensor unit, for instance for monitoring one or more physiological parameters, to continue to be used for an extended period by a patient at home after being discharged. A sensor patch of the system will often need to be replaced on a regular basis. When the patient positions the new patch, they may position it inaccurately. The system can determine an indication of position of the patch, and optionally may guide placement by a patient based on this. Over the period at home, the parameters may change. The re-calibration functionality hence allows these to be kept up to date.

FIG. 3 schematically depicts in block diagram form an example on-body sensing system 30 in accordance or more embodiments.

The system 30 comprises two skin interface units 32, 34 for electrically interfacing with the skin of a subject. A first skin interface unit 32 is adapted for coupling generated electrical signals into the body. A second skin interface unit 34 is for sensing said coupled signals at a remote location on the subject's skin. Although FIG. 3 shows the first and second interface units as being adjacent to one another, this is schematic only. In use, with the system positioned in situ on the body of a subject, the units are preferably located remote from one another on the body.

The system further includes a controller 36, operatively coupled with the skin interface units 32, 34. The controller is adapted to control signal generation and sensing using the skin interface units. As will be explained in greater detail below, the circuitry for generating signals and for processing received signals can be distributed in different ways between the components of the system. In some examples, this circuitry is all comprised by the controller. In other examples, the first skin interface unit may comprise local circuitry for generating electrical signals. In other examples, the signals may be generated externally, for instance by the controller.

In FIG. 3, the controller 36 is shown as a separate dedicated control unit. However, as noted above, in other examples, the control function may be performed by one or both of the skin interface units 32, 34 themselves. Hence, in the latter case, the controller is a distributed controller. Hence one or both of the skin interface units may comprise the controller in some examples, i.e. the control function is distributed among the skin interface units of the system. In the following description, reference to the controller 36 may be understood as referred either to a dedicated control unit or to one or more of the interface units of the system performing the relevant control function.

The system 30 may be a physiological parameter monitoring system. In particular, one or both of the skin interface units 32, 34 may be for sensing one or more physiological signals (such as electrocardiogram (ECG) or electromyography (EMG) signals for example). Accurate positioning of the skin interface unit(s) is in this case important in order that monitoring of these physiological parameters is accurate.

Partly to assist in this, the controller 36 is operable in one control mode to determine an indication of a position of one of the skin interface units 32, 34 based on sensed signal characteristics at the second unit 34 and one or more pre-determined body-transmission parameters, and based on a known location of the other of the units. The body transmission parameters may relate to properties of the body or skin as a medium for carrying electrical signals, i.e. the generated signals. They may additionally or alternatively relate to, and be derivable from, characteristics of the sensed signals (after their propagation through the skin or body).

The one or more body transmission parameters may include for example at least one of: signal wavelength, signal propagation velocity, phase angle difference (between two skin coupling electrode pairs on a single interface unit), signal transmission time (between generation and receipt), and signal attenuation (between generation and receipt).

As noted above, the body transmission parameters can change over time, e.g. due to changing moisture levels on the skin. This means that unless the parameters are updated, position determinations will be inaccurate. To overcome this, the controller 36 is operable in a further control mode to perform a re-calibration procedure for re-determining said body-transmission parameters based on a known initial position of both of the interface units 32, 34.

This procedure comprises in summary the following steps. The first skin interface unit 32 is controlled to apply or couple one or more reference signals to the body or skin. The unit may generate these or they may be generated externally and output to the unit for application to the body.

The generated reference signals are sensed at the second skin interface unit 34 and at least one of the body-transmission parameters is re-determined based on the sensed signal characteristics and on said known initial position of the units 32, 34.

Finally, the pre-determined body-transmission parameters are then corrected or updated, for instance in a local memory or remote data store, based on any differences between the at least one re-determined parameter and the corresponding pre-determined parameter. If there are no differences, no correcting need be performed.

The known initial position of at least one of the skin interface units 32, 34 (in particular the one having a position which is determinable by the controller) may be a position previously determined by the controller and for example stored in a memory. This prior determined position may be a position determined by the controller a certain threshold time in the past, for instance at least an hour in the past or more preferably at least a day in the past.

Alternatively, the known initial position may be a pre-set position, for instance stored in a memory. For example, a clinician may initially place the skin interface unit in this pre-set position using their expert knowledge. For the re-calibration procedure, the unit may be assumed to be in this pre-set position.

Subsequently, when the unit is re-positioned, the user may be guided in re-placing the unit in this same pre-set position, or a different pre-set position, based on a real-time determination of a current indication of position, and optionally a comparison of this to the pre-set position indication or to a set of pre-set position indications.

One of the skin interface units 32, 34 has a stable, known position, and the controller is operable to determine a position of the other. Preferably, the first interface unit 32 (the signal transmitter unit) has a known location, and the controller is operable in the one control mode to determine a position of the second interface unit 34.

Preferably, the re-calibration procedure is based on this known location of the first interface unit, and a known initial position of the second interface unit.

The skin interface units can take different forms.

In a preferred set of embodiments, one or both of the skin-interface units is in the form of a body-mountable unit, for example a sensor patch or wearable device.

The first interface unit 32 is preferably an on-body unit for mounting against a pre-determined region of the skin of the subject, or an off-body unit for placement against a pre-determined region of the skin of the subject.

For example, the first interface unit may be a wearable unit configured for mounting to a particular part of the body. In advantageous examples, the first interface unit is in the form of a wrist mountable device. This carries the advantage that the position of the first interface unit in this case is stable and reliably known. However, this effect could also be achieved with other body-mounted devices that can be fixedly secured to a part of the body, e.g. a chest strap, ankle band or ear hook by way of example.

The first interface unit may be in the form of a smart watch device. The smart watch device may comprise the controller 36 according to examples.

Preferably, the second skin interface unit 34 (for sensing) is in the form of a sensor patch or pad for mounting against the skin of the subject. The sensor patch may comprise a flexible electrode for sensing electrical signals from the skin or body. The patch may have an adhesive layer for coupling the patch to the skin.

The second interface unit 34 may be for use also in monitoring one or more physiological parameters as part of a physiological parameter monitoring function of the system 30. The physiological parameters may be vital signs for instance.

The handling of signal generation and processing can be distributed between components of the system in different ways. In one set of examples, signal generation and processing of received signals may be performed centrally by a central controller 36, wherein the first and second skin interface units are simply for electrically coupling generated and received signals into and back out from the body. They may each simply comprise one or more electrodes for facilitating this for instance.

Alternatively, signal generation and the processing of received signals may be distributed between the skin interface units. For example, the first skin interface unit 32 may comprise circuitry for generating the signals for applying to the body, and the second skin interface unit may comprise circuitry for processing the sensed signals.

In further examples, both the first and second skin interface units may each be selectably configurable either as signal generator (i.e. transmitter) or as signal sensor (i.e. receiver). Each may comprise circuitry both for generating signals for coupling into the body and for processing signals coupled back out of the body. Each skin interface unit may be switchable between the two modes or functionalities, to thereby increase flexibility of the system. Such a skin interface unit may be termed a multi-function interface unit.

FIG. 4 shows in block diagram form circuitry which may be comprised by such a multi-function skin interface unit, to permit implementation of both signal generation and processing of received signals.

The circuitry together forms a transceiver unit 42 for controlling generation and transmission of signals through the body via the given skin interface unit, and also for receiving of signals at a remote location via a second skin interface unit. This transceiver unit may be referred to as an RF unit.

The transceiver unit 42 includes in this example one set of components for controlling signal generation and transmission (transmitter part 44) and a second set of components for controlling receiving of signals (receiver part 46). Both parts are operatively connected to a microcontroller unit (MCU) which controls the transmitter 44 and receiver 46 parts. Both the transmitter and receiver parts are connected with a switch 50 which interfaces with pair of skin contacting electrodes 51a, 51b, labelled electrode A1 and A2. The switch is for switching the given interface unit 32, 34 between signal transmission mode (which connects the electrodes to the transmitter part 44) and signal receiving mode (which connects the electrodes to the receiver part 46).

The signal transmission part 44 includes a signal generator 56 adapted to generate electrical signals for coupling into the skin by the electrodes 51. The signal generator advantageously generates alternating signals at radio frequencies. Preferably, signals are generated in a frequency range 10 MHz to 150 MHz as in this frequency range the human body behaves as a waveguide for signal transmission.

The transmitter part 44 further includes a voltage booster and driver 54 adapted to receive the generated raw signals, amplify (i.e. boost) them and drive application of the signals, via the switch and electrodes 51, to the body.

The signal receiver part 46 includes an analog front end element 60 for receiving in analogue form, via the switch 50, the raw signals sensed by the electrodes 51. The front end element communicates the received signals to an analogue-to-digital converter 62 which processes the signals and outputs them in digital form to the microcontroller unit 48.

In other examples, each of the skin interface units 32, 34 may be configured to perform only one of signal generation or signal sensing. In this case, each may comprise only one of the transmitter 44 or receiver 46 parts shown in FIG. 4, and the switch may be omitted. For instance, the first skin interface unit 32 may comprise the transmitter part 44, and the second skin interface unit 46 may comprise the receiver part 46.

In further examples, both the transmitter 44 and receiver 46 part of the transceiver unit 42 shown in FIG. 4 may be comprised by the central controller 36, with the controller 36 configured to electrically communicate signals to and from the skin interface unit.

The illustrated transceiver unit 42 shown in FIG. 4 represents one example only of circuitry which may be used to generate and process signals in accordance with embodiments of the invention. Other suitable circuitry implementations, capable of achieving similar functionality, will be apparent to the skilled person.

To illustrate the concept of the invention, one advantageous embodiment will now be described in detail, by way of example only.

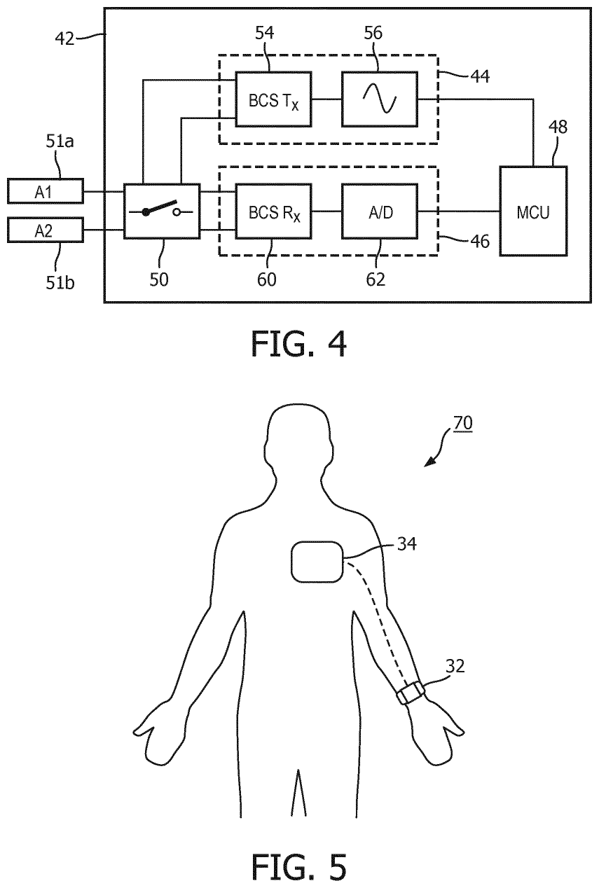

A layout of the system 30 according to this embodiment is illustrated schematically in FIG. 5. The system is illustrated in situ with components mounted on the body of a subject 70. The system comprises a first skin interface unit 32 in the form of a smart watch device. The first skin interface unit is for coupling generated signals into the body. A second skin interface unit 34 is provided in the form of a sensor patch 34. The sensor patch is for sensing the signals coupled into the body by the smart watch device.

Although a smart watch is used, a different wearable device could be used in accordance with other examples of this embodiment. Alternatively again, an off-body device could be used such as a smart weight scale. This provides the same advantage that the location of the device on the body is reliably known, as it is configured for application to a particular location on the skin of the subject (i.e. the feet in this case).

The wearable device 32 and sensor patch 34 each comprise at least one pair of electrodes for directly interacting with the skin. Preferably these electrodes are configurable in each of the units to be transmitter electrodes (for applying signals to the body) or receiver electrodes (for sensing the applied signals). In this way, the wearable device and patch can be selectively configured as transmitter or receiver respectively.

In this case, both the wearable device 32 and sensor patch 34 each comprise a transceiver circuit 42 as illustrated in FIG. 4 and described above. As described above, this includes both a body channel signal (BCS) transmitter 44 and a body channel signal (BCS) receiver 46.

The system may further comprise a dedicated controller (not shown). Alternatively, the control function may be performed by one or both of the skin interface units. For example, the controller may be comprised by the wearable device 32. Reference to a controller may be taken as referring to either option.

Communication between the patch 34 and wearable device 32 may be facilitated via any suitable communication medium or channel, either wired or wireless. For reasons of comfort and flexibility, wireless communication may be preferred. Both the wearable device 32 and patch 34 may in this case comprise wireless communication modules to facilitate this. These may comprise standard communication technologies such as Bluetooth, Wi-Fi, ultra-wide band (UWB) or body-coupled communication for instance.

The controller is configured in one control mode to determine a position and orientation of the patch 34. To determine the correct position and orientation of the patch on the body, a transmitter (patch or the wearable device) transmits a signal from a multitude of transmitting electrodes which is then received by a multitude of receiving electrodes on the receiver (wearable device or patch).

A number of transmission parameters of the received signals are sensed or determined based on measured signal characteristics of the received signals. These may include for instance signal transmission time (between the transmitter and receiver), signal attenuation (between transmitter and receiver) and phase angle difference (between at least two electrode pairs comprised by the second interface unit). A number of pre-determined body transmission parameters are stored in a memory of the controller or stored remotely.

Preferably, these body transmission parameters are pre-determined signal transmission parameters of the same variety as those derived for the signals received at the sensor patch 34, and each pertaining to signals corresponding to a different known position of the patch relative to the wearable device. In particular, preferably, there are stored a plurality of sets of transmission parameters corresponding to various possible correct positions and orientations of the patch. In this way, by comparing the measured parameters with these pre-determined parameters, it can be determined whether the current position of the patch matches any of the various correct positions to which the pre-determined parameters correspond. If not, output information may be generated and communicated to the user to indicate that there is no match, or more preferably to provide instructions to the user as to how to move the patch so as to approach a correct positioning. This may be via a sensory output device such as a display (e.g. on the wearable device 32) or a speaker, or a haptic output device (e.g. vibration of the wearable device). If there is a match, output information may be generated and communication to the user representative of this.

Alternatively, generalized body transmission parameters may be stored, corresponding for instance to general characteristics of signal transmission through the body as a signal carrying medium. These may include for instance, signal velocity and signal wavelength. These general parameters can be used to determine from the specific signal characteristics (e.g. transmission time, attenuation, phase angle difference) a distance or separation between the transmitter 32 and receiver 34.

As noted above, in some cases the body transmission parameters are signal transmission parameters derivable from measured signal characteristics, and with different sets of the pre-determined parameters corresponding to a different unique relative positioning of the two units. In this case, the transmission parameters may include one or more of: phase angle difference between two electrode pairs on a given skin interface unit, signal transmission time between transmission and receipt of a signal and signal attenuation between transmission and receipt of a signal.

Transmission time means signal time of flight: propagation duration between initial transmission and receipt. Signal attenuation may mean signal path loss: change in signal strength between initial transmission and receipt. Other signal characteristics may additionally or alternatively be derived.

Means for deriving these parameters based on measurable signal characteristics will now be described. The descriptions apply with full generality, hence for greatest clarity, the first skin interface unit 32 (for transmitting signals) will simply be referred to as the transmitting unit 32, and the second skin interface unit 34 (for receiving signals) will be referred to as the receiving unit 34.

Deriving the phase angle of a sensed signal is a standard procedure and the skilled person will be aware of means for implementing this functionality.

Deriving the signal time of flight (transmission time) can be achieved simply by recording the time of transmission of the signal and the time of receipt of the signal and calculating the difference. For performing this, the transmitting unit 32 and the receiving unit 34 may each comprise an internal clock and the clocks may be synchronized. Alternatively a central controller 36 may track the time of transmission of the signal and receipt of the same signal at the receiving unit. Directly sequential transmission and receipt events may be assumed to be associated with the same signal.

Deriving the path loss (signal attenuation) may be achieved simply by recording the signal strength at transmission (or generating the signal for transmission at a known strength) measuring the strength of the same signal on receipt, and then computing the change. The strength of the signal may refer for example to signal amplitude, e.g. in volts, for example peak-to-peak amplitude. The receiving unit 34 and the transmitting unit 32 may each comprise signal processing means permitting measurement of the signal strength, e.g. signal amplitude. In this case, each of the units may comprise a clock, and the clocks may be synchronized, allowing the transmission and receipt of a given signal to be matched to one another. In particular, directly sequential transmission and receipt events may be assumed to be associated with the same signal. However, alternatively a central controller may comprise signal processing means for measuring the signal strength of signals received at the receiving unit, e.g. amplitude, and may be configured to calculate a signal attenuation between transmission and receipt.

In advantageous examples, the receiving unit 34 (the second skin interface unit) comprises two or more pairs of electrodes. In this case, advantageously, one or more differential transmission parameters may be derived, corresponding to a difference in the value of a given transmission parameter between two pairs of electrodes of the unit 34.

For example the differential transmission parameter may be one or more of: phase angle difference, signal transmission time (time of flight) difference and signal attenuation (path loss) difference. In each case the difference is between the value as measured at each of two electrode pairs of a receiving unit 34. The value of the differential transmission parameter for the receiving unit may be determined by a controller 36 for example, or may be determined locally by the receiving unit 34. Optionally, a differential value may be derived for one or more transmission parameters in respect of each and every combination of electrode pairs comprised by the receiving unit (where there are more than two).

A differential transmission parameter provides a particularly precise characterization of positioning, and in particular orientation, since the difference in the measured values at two spatially separated electrode pairs varies consistently depending on orientation state.

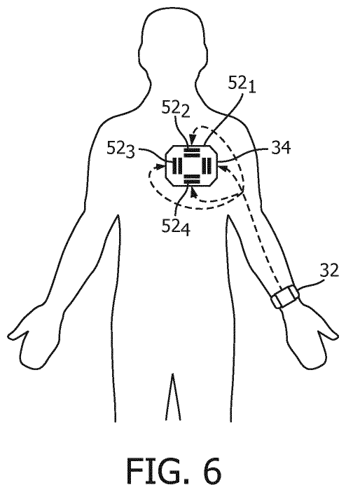

To illustrate, FIG. 6 shows an example receiving unit 34 comprising four pairs of electrodes 52₁, 52₂, 52₃, 52₄, although the concept can also be applied with fewer than four pairs, e.g. two pairs or three pairs. The receiving unit is shown positioned in situ on the body, along with a transmitting unit 32 in the form of a wrist-mounted device. All possible phase angle differences between the signal that is received at the at least two electrode pairs of the receiving unit can be described as follows:

Øᵢⱼ=Øᵢ−Øⱼ,

which indicates the phase angle difference Ø between the signal received at electrode pairs 52₁ and 52ⱼ respectively. Accordingly, all phase angle differences Øᵢᵢ between an electrode pair 52₁ and itself, where i=1, 2, 3, 4, is zero. The phase angle difference Øᵢⱼ between electrode pairs 52ᵢ and 52ⱼ respectively is the same but the opposite sign to the phase angle difference Øⱼᵢ between electrode pairs 52ⱼ and 52ᵢ, i.e. Øᵢⱼ=−Øⱼᵢ, where i=1, 2, 3, 4 and j=1, 2, 3, 4 and when i≠j.

For example, Ø₁₂=Ø₁−Ø₂ indicates the phase angle difference between the signal received at the electrode pairs 52₁ and 52₂, and so on. Thus, in an embodiment where the receiving unit 34 is orientated as shown in FIG. 6, the phase angle difference Ø₁₃ between the signal received at electrode pairs 52₁ and 52₃ respectively will be negative and the phase angle difference Ø₃₁ between the signal received at electrode pairs 52₃ and 52₁ respectively will be positive indicating that electrode pair 52₁ is closer to the transmitting unit 32 than the electrode pair 52₃.

The phase angle difference Ø₂₄ between the signal received at electrode pairs 52₂ and 52₄ and the phase angle difference Ø₄₂ between the signal received at electrode pairs 52₄ and 52₂ will be zero (or almost zero) due to an equal (or almost equal) distance between the electrode pairs 102₁ and 102₄ and the transmitting unit 32. The phase angle difference Ø₁₂ between the signal received at electrode pairs 52₁ and 52₂ and phase angle difference Ø₁₄ between the signal received at electrode pairs 52₁ and 52₄ will be small but negative and phase angle difference Ø₃₂ between the signal received at electrode pairs 52₃ and 52₂ and the phase angle difference Ø₃₄ between the signal received at electrode pairs 52₃ and 52₄ will be small but positive. These phase angle differences thus characterize in a precise way the orientation of the receiving unit 34 with respect to the transmitting unit 32.

As discussed, another possible differential transmission parameter which may additionally or alternatively be derived is a time of flight (ToF) of the signal received at one of the at least two electrode pairs 52₁, 52₂, 52₃, 52₄ of the receiving unit 34 relative to a time of flight of the signal received at least one other of the at least two electrode pairs 52₁, 52₂, 52₃, 52₄ of the receiving unit 34. The relative time of flight of the signals is also indicative of an orientation of the receiving unit with respect to the transmitting unit 32. More specifically, the longer the time of flight of the signal received at an electrode pair 52₁, 52₂, 52₃, 52₄, the further away the electrode pair is from the transmitting unit. Similarly, the shorter the time of flight of the signal received at an electrode pair, the closer the electrode pair is to the transmitting unit.

For example, in an example where the receiving unit 34 is orientated as shown in FIG. 6, the time of flight t₁ from the transmitting unit 32 to the electrode pair 52₁ is lowest compared to the time of flight t₂, t₃ and t₄ from the transmitting unit 32 to the electrode pairs 52₂, 52₃ and 52₄ respectively. The time of flight t₃ from the transmitting unit 32 to the electrode pair 52₃ has the highest value, while the time of flight t₂ and t₄ from the transmitting unit 32 to the electrode pairs 52₂ and 52₄ respectively will be equal (or almost equal) and less than the time of flight t₃ from the transmitting unit 32 to the electrode pair 52₃ but greater than the time of flight t₁ from the transmitting unit 32 to the electrode pair 52₁.

Thus, where the receiving unit 34 is orientated as shown in FIG. 6, the relative time of flight of the signals can be described as follows:

t₁≤t₂,t₄≤t₃.

This can provide information in particular on the orientation of the receiving unit 34 with respect to the transmitting unit 32. In some embodiments where the property is a time of flight (ToF), the receiving unit 34 may be time synchronized with the transmitting unit 32 prior to the transmission of the signal from the transmitting unit 32 (for example, in the manner described earlier). In these embodiments, the receiving unit 34 may generate a reference signal using an internal synchronized clock of the receiving unit 34. The reference signal can provide a reference to the signal transmitted from the transmitting unit 32. Alternatively a central controller 36 keeps track of the times of transmission and receipt.

As discussed, a transmission parameter which may additionally or alternatively be derived is the amplitude of the signal received at one of at least two electrode pairs 52₁, 52₂, 52₃, 52₄ relative to an amplitude of the signal received at least one other of the at least two electrode pairs 52₁, 52₂, 52₃, 52₄. In these examples, the relative amplitude of the signals may be indicative in particular of the orientation of the receiving unit 34 with respect to the transmitting unit 32.

Due to the impedance of the body, the signal transmitted from the transmitting unit 32 undergoes attenuation as it travels through the body. Thus, the amplitude of the signal transmitted from the transmitting unit 32 decreases as it travels through the body. Attenuation of the signal occurs. The longer the signal travels through the body, the more the signal is attenuated (or the more the amplitude of the signal decreases). Thus, the lower the amplitude of the signal received at an electrode pair 52₁, 52₂, 52₃, 52₄ of the receiving unit 34, the further away the electrode pair 52₁, 52₂, 52₃, 52₄ is from the transmitting unit 32. Similarly, the higher the amplitude of the signal received at an electrode pair of the receiving unit 34, the closer the electrode pair is to the transmitting unit 32.

For example, in an example where the receiving unit 34 is orientated as shown in FIG. 6, the amplitude of the signal received at the electrode pair 52₃ is lowest compared to the amplitude of the signal received at the other electrode pairs 52₁, 52₂ and 52₄. Similarly, the amplitude of the signal received at the electrode pair 52₁ is highest compared to the amplitude of the signal received at the other electrode pairs 52₂, 52₃ and 52₄. The amplitude of the signal received at the electrode pairs 52₂ and 52₄ is equal (or almost equal) and less than the amplitude of the signal received at the electrode pair 52₁ but greater than the amplitude of the signal received at the electrode pair 52₃.

In some embodiments, the signal attenuation G may be derived as follows:

G=20 log₁₀(V_{receive}/V_{send}),

where V_{receive} is the amplitude of the signal received at the at least two electrode pairs 52₁, 52₂, 52₃, 52₄ of the receiving unit 34 and V_{send} is the amplitude of the signal transmitted from the transmitting unit 32.

The greater the signal attenuation of the signal received at an electrode pair 52₁, 52₂, 52₃, 52₄ of the receiving unit 34, the further away the electrode pair is from the transmitting unit 32. Similarly, the lesser the attenuation of the signal received at an electrode pair of the receiving unit 34, the closer the electrode pair 52₁, 52₂, 52₃, 52₄ is to the transmitting unit 32. Thus, in an embodiment where the receiving unit 34 is orientated as shown in FIG. 6, the attenuation of the signal received at the electrode pair 52₁ is greater than the attenuation of the signal received at the electrode pair 52₂ and the attenuation of the signal received at the electrode pair 52₄ is less than the attenuation of the signal received at the electrode pair 52₃.

The above explanations represent just one set of example of transmission parameters which may be derived and do not limit the invention. Advantageously both differential transmission parameters (differences in transmission parameters values between two pairs of electrodes of the receiving unit) are calculated in addition to absolute values of the same transmission parameters. The latter is particularly useful for characterizing and thus deriving position of a receiving unit (the second skin interface unit). The former is especially useful for characterizing orientation.

The system 30 according to embodiments of the invention is aimed in particular at addressing the issue of accurate re-application of the patch 34 by the patient at home after it has initially been applied at a hospital or care unit by a trained nurse or other hospital personnel.

To facilitate this, the system 30 according to preferred embodiments may be adapted to facilitate a multi-stage configuration procedure comprising an initial placement and calibration procedure part, carried out by a trained clinician using the system, and a subsequent re-calibration and re-positioning procedure, carried out by the patient when at home using the system.

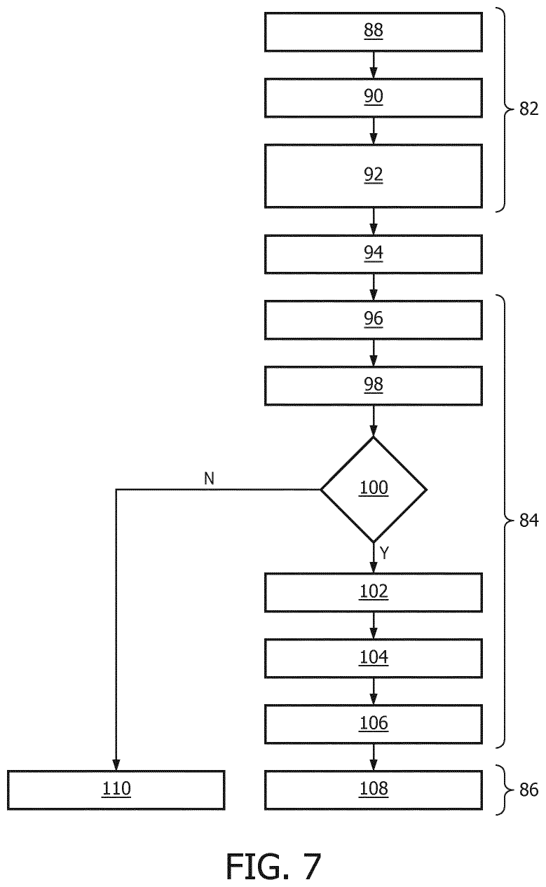

An example of this multi-stage procedure will now be described. Steps of the procedure are set out in block diagram form in FIG. 7.

First, an initial calibration procedure 82 is performed to determine and store the predetermined transmission parameters discussed above, based on at least one known initial location of each of the skin-interface units. More particularly, and as will be explained below, preferably the initial calibration process comprises determining and storing a plurality of sets of transmission parameters, each set corresponding to a different particular position and optionally orientation of one of the skin interface units, for a known static position of the other.

During the patch calibration procedure 82, the nurse or other care personnel indicates 88 to the controller of the system that calibration is to take place. This may for instance be by running or activating a particular application on the wearable device or other external controller for configuring the patch. It may be by otherwise switching the controller or wearable device into a specific control mode.

The clinician then takes a patch and places it at various different possible correct positions and orientations on the patient's body (90). By correct is meant positions which permit accurate sensing and monitoring of the particular physiological parameter(s) which are to be monitored using patch when the patient is at home.

For each of these correct patch positions and orientations, the controller controls the wearable device and patch to generate and sense signals respectively. Based on sensed signal characteristics at the patch, a list of transmission parameters of interest (PoI) are determined 92 for the given position, in accordance with the procedure(s) set out above. These determined parameters may be represented for each correct position as follows:

POI_{i,orig_j}={POI_{1,orig_j},POI_{2,orig_j},POI_{3,orig_j},POI_{4,orig_j},POI_{5,orig_j},POI_{6,orig_j}, . . . POI_{i,orig_j}},i€{1,2, . . . i},j€{1,2, . . . j}

The subscript orig_j denotes that the calculated list of PoIᵢ parameters corresponds to the jth one of the correct positions and orientations of the patch on the body when the nurse/care personnel applies the patch on the patient's body at the hospital/care center. The index i corresponds to the numerous parameters for each different position.

The caregiver may for instance provide an indication to the controller when the patch has been moved to each new position (e.g. using a user interface), so that the controller may then calculate the corresponding set of transmission parameters.

Once transmission parameters for each of the correct positions have been calculated, these parameters are stored by the system. This may be in a local memory of the system, for instance comprised by the controller and/or wearable device and/or patch, or remotely, for instance in the cloud or other remote data store.

After the initial calibration procedure 82, the nurse or other care personnel attaches the patch 94 at one of the possible correct positions and orientations on the patient's body. The particular position and orientation at which the patch is attached is recorded, locally or remotely.

The patient may then be discharged from the hospital or care center. Once at home, the patch typically needs replacing every two to three days. This requires removing the patch and re-attaching a new patch. When the patient re-applies the new patch, the system provides guidance as to its placement based on determining a current position, which determination (in this example) requires deriving signal transmission parameters and comparing these to the stored transmission parameters for the various correct positions. This process will only work accurately if the stored parameters are still accurate. However since these parameters are dependent upon skin moisture levels, they change over time for the same given positions. Hence re-calibration is required.

Hence, before removing the patch from its current position and orientation, a re-calibration procedure 84 is triggered. This may be done for example by initiating 96 a patch re-calibration routine on an app running on the wearable device or other controller, or otherwise indicating to the system controller that re-calibration is to be performed.

Once the re-calibration procedure has been triggered, the system 30 re-determines the PoI transmission parameter values for the single position in which the patch is still applied.

The system then compares 100 the re-determined parameters with the PoI_{orig} parameter values calculated for the same given position when the nurse originally calibrated the system, and determines whether they are different. The particular one of the positions to which the new parameters corresponds is known because it is stored when the nurse originally attaches the patch at the hospital.

Due to changes in the skin properties, the body channel during patch re-application may often be different from the channel during patch application. As a consequence, the new set of PoI_{new} values may differ from PoI_{orig} values stored for this given position.

If it is determined that there are differences, a correction factor is computed 102 based on the comparison 100. The correction factor may be denoted by κ=f(PoI_{orig}, PoI_{new}).

Various options exist for computing the correction factor. It may for example be a simple ratio of PoI_{orig} and PoI_{new} or may be a more complex function.

This correction factor is then applied 104 to the whole set of stored, pre-determined PoI_{j, orig} values for all of the possible patch positions and orientations calculated during the initial calibration procedure. This then yields a set of corrected parameter values which are then stored 106 locally at the patch, wearable device or other controller, or stored remotely, e.g. in the cloud or other remote data store. The corrected parameter values for each of the possible correct positions are stored in place of the original values, thereby updating them.

The system 30 may provide a sensory output to indicate to the patient or user that the recalibration procedure 84 is complete.

The patient may then remove the current patch, and replace it with a new one in a replacement procedure 86. Depending upon where the new transmission parameters are stored, this may require transferring the new parameters to the new patch. When the patient attaches the new patch, the system uses the corrected body-transmission parameters to provide assistance 108. This is based on determining an indication of the position of the patch, which is done using the newly computed parameters.

In particular, according to the present example, signal transmission parameters are derived for the current position, and these compared with the stored parameters to see whether the parameters for the current position match any of those stored for the correct positions. If not, the system derives that the patch is placed in a position away from a correct position and may indicate this to the user e.g. with a sensory output) If there is a match, the system derives that the patch is in a correct position. In this way, guidance can be provided to assist in placing the patch in one of the correct positions and orientations.

Once the patch is placed in one of the correct positions, the particular one of the positions in which it is placed is determined (by the comparison of the transmission parameters) and stored.

If, when comparing 100 the newly calculated parameter values PoI_{new} with the original values PoI_{orig}, there is no difference, then the original values are retained, and the patch may be removed and replaced 110, with positioning guidance provided based on the original parameter values.

Although the above procedure has been described with reference specifically to a patch and wearable device, the same procedure may be implemented for a system 30 comprising any first and second skin interface unit.

As discussed above, embodiments may make use of a dedicated controller 36. The controller can be implemented in numerous ways, with software and/or hardware, to perform the various functions required. A processor is one example of a controller which employs one or more microprocessors that may be programmed using software (e.g., microcode) to perform the required functions. A controller may however be implemented with or without employing a processor, and also may be implemented as a combination of dedicated hardware to perform some functions and a processor (e.g., one or more programmed microprocessors and associated circuitry) to perform other functions.

Examples of controller components that may be employed in various embodiments of the present disclosure include, but are not limited to, conventional microprocessors, application specific integrated circuits (ASICs), and field-programmable gate arrays (FPGAs).

In various implementations, a processor or controller may be associated with one or more storage media such as volatile and non-volatile computer memory such as RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into a processor or controller.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. An on-body sensor system, comprising:
at least two skin interface units for electrically interfacing with the skin of a subject, including a first unit for coupling generated signals into the body, and a second unit for sensing said coupled signals at a remote location on the subject's skin, one of the units for placement at a known location on the body; and
a controller adapted to control signal generation and sensing using the skin interface units, and operable in one control mode to determine an indication of a position of one of the units based on sensed signal characteristics at said second unit and one or more pre-determined body-transmission parameters;
wherein the controller is operable in a further control mode to perform a re-calibration procedure for re-determining said body-transmission parameters based on a known initial position of the two units, the procedure comprising:
controlling the first skin interface unit to generate one or more reference signals,
sensing the reference signals at the second skin interface unit and re-determining at least one of the body-transmission parameters based on the sensed signal characteristics and said known initial position of the two units, and
correcting the pre-determined body-transmission parameters based on any differences between the at least one re-determined parameter and the corresponding pre-determined parameter.

2. The on-body sensor system as claimed in claim 1, wherein the first interface unit has a known location, and the controller is operable in the one control mode to determine a position of the second interface unit, and wherein the re-calibration procedure is based on the known location of the first unit, and a known initial position of the second unit.

3. The on-body sensor system as claimed in claim 1 wherein one or both of the skin-interface units is in the form of a body-mountable unit.

4. The on-body sensor system as claimed in claim 2, wherein the first interface unit is an on-body unit for mounting against a pre-determined region of the skin of the subject, or an off-body unit for temporary placement against a pre-determined region of the skin of the subject.

5. The on-body sensor system as claimed in claim 2, wherein the first interface unit is a wearable unit configured for mounting to a particular part of the body.

6. The on-body sensor system as claimed in claim 1, wherein at least the second skin interface unit is in the form of a sensor pad for mounting against the skin of the subject.

7. The on-body sensor system as claimed in claim 1, wherein the one or more body transmission parameters include at least one of: signal wavelength, signal propagation velocity, phase angle difference between two electrode pairs on a given skin interface unit, signal transmission time between transmission and receipt of a signal, signal attenuation between transmission and receipt of a signal.

8. The on-body sensor system as claimed in claim 1, wherein the pre-determined transmission parameters comprise a plurality of sets of pre-determined transmission parameters, each set corresponding to a different particular position of the skin interface unit whose position the controller is operable to determine.

9. The on-body sensor system as claimed in claim 1, wherein the system is for monitoring one or more physiological parameters of a subject, and wherein the first interface unit is for use in sensing the one or more physiological parameters.

10. The on-body sensor system as claimed in claim 1, wherein correcting the pre-determined body-transmission parameters comprises:
comparing the at least one re-determined body-transmission parameter with the corresponding pre-determined parameter and determining a parameter correction factor based on the comparison;
applying the correction factor to each of the pre-stored transmission parameters in order thereby to correct the parameters, and
storing the corrected parameters in place of the pre-determined parameters.

11. The on-body sensor system as claimed in claim 1, wherein the controller is further adapted in accordance with one control mode to perform an initial calibration procedure to determine and store said predetermined transmission parameters, based on measured signal characteristics at the second skin interface unit with the two units placed in at least one known set of locations.

12. The on body sensor system as claimed in claim 11, wherein the initial calibration procedure comprises determining and storing a plurality of sets of transmission parameters, each set corresponding to a different particular position and optionally orientation of the skin interface unit whose position the controller is operable to determine.

13. The on-body sensor system as claimed in claim 1, wherein the controller is adapted in accordance with one control mode to guide a user in positioning one of the interface units based on determining an indication of a current position of the unit using the sensed signal characteristics and body-transmission parameters.

14. A method of configuring an on-body sensor system, the system comprising at least two skin interface units for electrically interfacing with the skin of a subject, including a first unit for coupling generated signals into the body, and a second unit for sensing said coupled signals at a remote location on the subject's skin, one of the units for placement at a known location on the body,
and the system operable to determine an indication of position of one of the units based on sensed signal characteristics at said second unit and one or more pre-determined body-transmission parameters,
and the method comprising
executing a re-calibration procedure for re-determining the one or more body-transmission parameters based on a known initial position of the two units, the procedure comprising
controlling the first skin interface unit to generate one or more reference signals, and sensing the reference signals at the second skin interface unit,
re-determining at least one of the body-transmission parameters based on the sensed signal characteristics and said known initial position of the two units, and
correcting the pre-determined body-transmission parameters based on any differences between the at least one re-determined parameter and the corresponding pre-determined parameter.

15. The method as claimed in claim 14 wherein the method further comprises, subsequent to the re-calibration procedure,
re-positioning one of the interface units on the body, and
determining a position of the re-positioned interface unit based on signal characteristics sensed at the second interface unit and the corrected body-transmission parameters.

16. The on-body sensor system as claimed in claim 5, wherein the first interface unit a wrist mountable unit.

17. The on-body sensor system as claimed in claim 8, wherein each set further corresponds to a different orientation of the skin interface unit.
